(19) United States
(12) Patent Application Publication
(10) Pub. No.: US 2006/0269574 A1
(43) Pub. Date: Nov. 30, 2006

(54) Method of repairing tendons by surgery

(76) Inventors: Johann Frederick De Beer, Cape Town (ZA); Polydoor Emile Huijsmans, Oegstgeest (NL)

Correspondence Address: MEYERTONS, HOOD, KIVLIN, KOWERT & GOETZEL, P.C., 700 LAVACA, SUITE 800, AUSTIN, TX, 78701, US

(21) Appl. No.: 11/140,776

(22) Filed: May 31, 2005

(51) Int. Cl. A61K 39/08; A61B 17/00

(52) U.S. Cl. 424/239.1; 606/1

(57) ABSTRACT

A method of repairing tendons by surgery is provided in which the muscle associated with the tendon to be repaired is treated with a predetermined dose of a neurotoxin sufficient to effectively cause temporary paralysis of the muscle. Any other muscle inextricably linked to that tendon may also be subjected to temporary paralysis. A sufficient period of time is allowed to elapse to enable said temporary paralysis of the muscle or muscles to set in prior to performing the necessary repair surgery on the relevant tendon in substantially conventional manner. The temporary paralysis of the relevant muscle or muscles obviates the necessity for postoperative immobilisation, typically by a sling or plaster cast, and ensures that the patient is not able to contract the relevant muscles during rehabilitation thereby possibly deleteriously affecting the repair.

### FIELD OF THE INVENTION

This invention relates to a method and preparation for repairing tendons by surgery and, more particularly, to a method that covers the extended procedure including preoperative preparation and postoperative rehabilitation.

It is to be understood that, in this specification, the term surgery is used in a broad sense and, of course, includes open surgery as well as the procedure known as arthroscopic surgery.

### BACKGROUND TO THE INVENTION

The surgical repair of tendons is typically accompanied by significant immobilisation following on the surgery and pending healing of the repaired tendon, typically for a period of the order of six weeks. Different forms of immobilisation are generally employed such as a sling in the case of a rotator cuff tendon repair and a plaster cast in the case of an Achilles tendon repair. In either case, and more particularly in the case of the use of a sling, rehabilitation is often accompanied by significant pain. The infliction of pain, in turn, results in involuntary muscular movements that are deleterious to, and consequently prolong, the healing process. Indeed, active motion or too much tension on the repaired tendon increases the risk of failure of the surgery.

It is noteworthy that many observers have found that it is virtually impossible, even when immobilising the arm with a sling or when immobilising the ankle in a plaster cast, to prevent contractions of the repaired muscle tendon units. The reason is that a person often involuntarily contracts muscles; a process that was learnt over many years and occurs when achieving balance moving in certain directions, for example.

As regards the repair of rotator cuff tendons specifically, applicant understands that of the order of 300,000 repairs take place annually in the United States of America alone. Each of these repairs is accompanied by the patient being subjected to considerable pain and, in many instances, worse still, considerable disability, during the rehabilitation period. With the patient having to be immobilised in a sling, for example, this greatly impacts on the independence of the person who is not able to care for himself or herself; not being able to drive a motorcar; and most importantly not being able to be economically active in many cases.

Relevant to this invention is the science of neurotoxins, the most well-known of which is botulinum toxin. This neurotoxin has been known for many decades and its medical uses, as far as applicant is aware, are restricted to the treatment of cervical dystonia; blepharospasm; strabismus; axilliary hyperhydrosis: spasticity in cerebral palsy; myofacial pain syndromes; and headaches. The dosages used in such applications are typically 5-10 units. It has also been used extensively in the field of cosmetics, particularly facial cosmetics, where the substance is injected into the facial muscles to lessen the degree of wrinkling caused by the “muscles of expression” in the face—the dosages used are typically 2-5 units.

It has apparently been used for postoperative treatment in cervical fusions in patients with cervical dystonia in order to reduce the tension in the fusion. It has also been proposed for use in treating muscle injuries (U.S. Pat. No. 6,423,391) and for the purpose of enhancing wound healing (U.S. Pat. No. 6,447,787) although applicant is unaware of any practical implementations of either of these proposals.

### OBJECT OF THE INVENTION

It is an object of this invention to provide a method of repairing tendons by surgery in which the pain suffered by patients can be reduced and rehabilitation greatly enhanced whilst generally obviating postoperative immobilisation. It is another object of the invention to provide a pharmaceutical preparation that is accompanied by information indicating its suitability for use in such a method.

### SUMMARY OF THE INVENTION

In accordance with one aspect of this invention there is provided a method of repairing tendons by surgery comprising the steps of
i identifying a tendon to be repaired;
ii identifying a muscle associated with that tendon and treating that muscle with a predetermined dose of a neurotoxin sufficient to effectively cause temporary paralysis of the muscle;
iii allowing a sufficient period of time to elapse to enable said temporary paralysis of the muscle to set in;
iv performing repair surgery on the relevant tendon; and,
v optionally, if necessary, treating said muscle with one or more additional doses of neurotoxin to maintain temporary paralysis for at least an initial period of rehabilitation.

Further features of the invention provide for the neurotoxin to be a slow acting neurotoxin, in particular, botulinum toxin, in which case the said sufficient period of time indicated in step iii is preferably of the order of 7 to 21 days and usually about 7-14 days and, in such an instance, for no additional doses of neurotoxin to be administered as envisaged in step v indicated above; for the degree of paralysis of the muscle to be tested immediately prior to performing the repair surgery, typically using a nerve stimulator; and for the results of the test for the degree of paralysis to be used as the basis for a decision on whether or not a particular patient will require any immobilisation post operatively.

One particularly important application of the present invention is to the repair of rotator cuff tendons in which case a tear to be repaired is generally of the tendon or tendons associated with the supraspinatus or infraspinatus muscles, or both, and in any event both of these muscles are preferably subjected to temporary paralysis preparatory to conducting the surgery irrespective of the fact that only the tendon associated with one of them is to be repaired. The reason for this is that even if only one of them is repaired, the other one could still contract and exert tension on the repair site as they are very close to each other and movement of one also generally causes motion of the tendon of the other.

In accordance with a second aspect of the invention there is provided in medical package comprising at least one dose of a neurotoxin in association with information indicating or describing its suitability for use in a method as defined above.

The invention is thus also directed at a neurotoxin for use in a method as defined above.

The overall effect of the invention is thus to temporarily paralyse the muscle of a repaired tendon thereby preventing its contraction with consequent pain to the patient; possible further muscular contractions following on the experiencing of pain; and the possibility of the repair being disturbed and possibly deleteriously affected.

In order that the invention may be more fully understood the application thereof to a rotator cuff tendon repair will be described in more detail below. In this description reference will be made to the accompanying drawings.

### BRIEF DESCRIPTION OF THE DRAWINGS

In the drawings:

FIG. 1 illustrates, schematically, the anatomy of a shoulder generally from the front thereof;

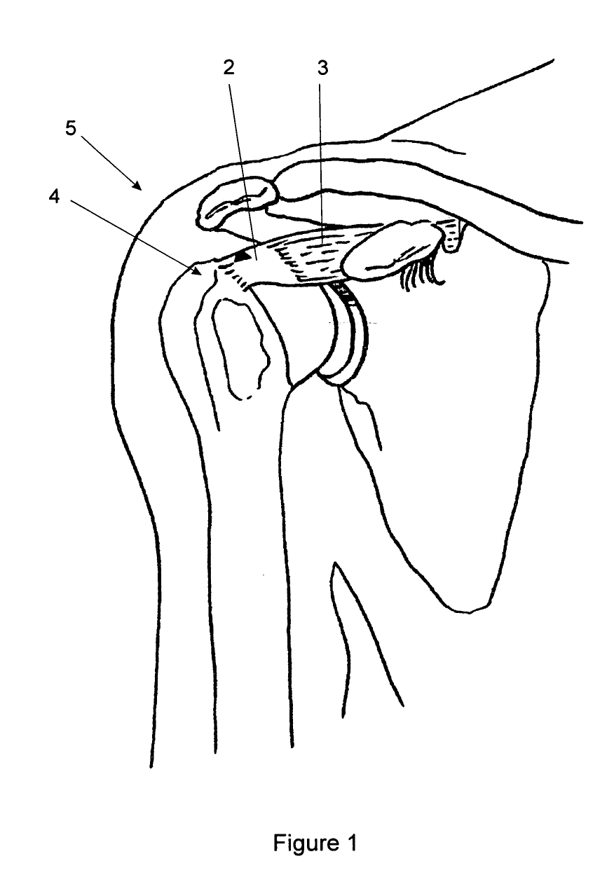

FIG. 2 illustrates, schematically, a shoulder generally from the front thereof and wherein a tear of the supraspinatus tendon is to be repaired and indicates one injection being instilled into one site in the supraspinatus muscle;

FIG. 3 illustrates the shoulder from the rear thereof and illustrates tears of both the supraspinatus and infraspinatus tendons of the affected shoulder and indicates one injection being instilled into one site in each of the supraspinatus and infraspinatus muscles;

FIG. 4 is similar to FIG. 1 but illustrates a repair having been performed on the supraspinatus tendon;

FIG. 5 illustrates, schematically, a shoulder generally from the front thereof and shows a preferred technique of injecting the supraspinatus muscle in multiple sites to ensure adequate paralysis; and,

FIG. 6 illustrates, schematically, a shoulder generally from the rear thereof and shows the preferred technique of injecting both the supraspinatus muscle and the infraspinatus muscle in multiple sites.

### DETAILED DESCRIPTION WITH REFERENCE TO THE DRAWINGS

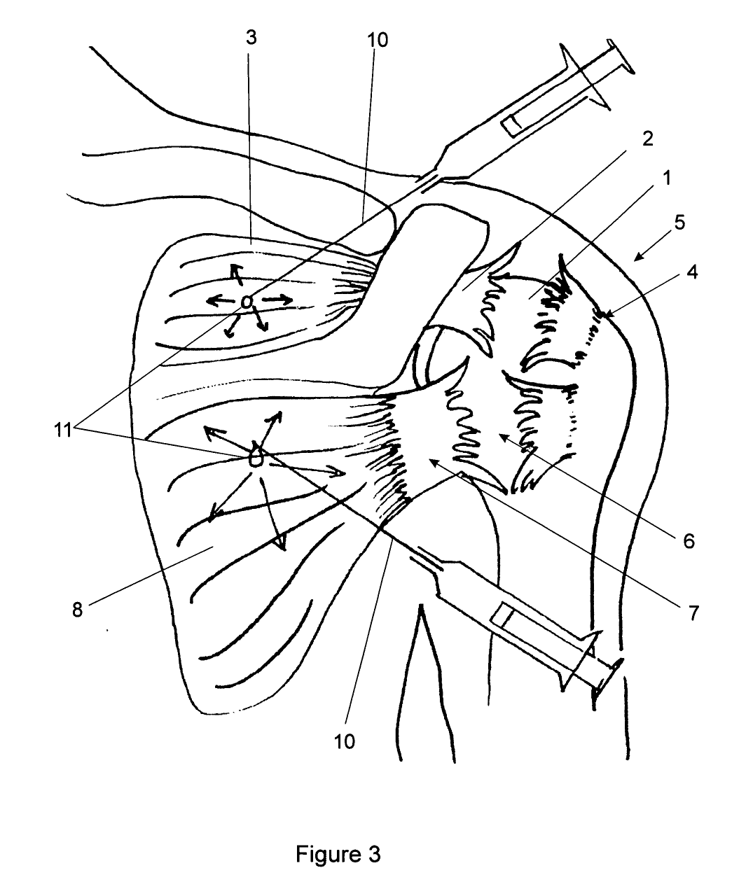

In this application of the invention a patient having a tear (1) in the tendon (2) attaching the supraspinatus muscle (3) to the greater tuberosity (4) of the shoulder (5) is treated according to the invention. Of course, it may be that a patient has a tear (6) in the tendon (7) attaching the infraspinatus muscle (8) to the greater tuberosity of the shoulder as well, or in the alternative. Such a tear is illustrated in FIG. 3.

In a preparatory phase of the repair procedure, both of the supraspinatus and infraspinatus muscles (3) and (7) of the affected shoulder (5) are treated with neurotoxin, in this case botulinum toxin type A that is sold under the trade name BOTOX™ by the American company Allergan Sales Inc of California. This treatment is scheduled suitably in advance of the planned surgery and typically between 7 and 21 days in advance, and most typically from 10 to 14 days in advance of the surgery.

Two vials of Botulinum toxin type A of 100 units each are used. One vial is used to treat the supraspinatus muscle and one vial to treat the infraspinatus muscle. Each vial is diluted to a total volume of 3.5 ml (about 0.21 cubic inches) with 0.9% saline solution.

These doses are required to ensure a complete paralysis of the muscles. This is considered to be safe as there are no other vital muscles in the immediate vicinity that may be endangered by the injections. The positioning of the injections is selected with the aim of optimising the effect of the neurotoxin to effectively paralyse the muscle without affecting any other muscles. Of course, the dose indicated above applies to an average adult having an average size of supraspinatus and infraspinatus muscles and could be varied according to muscle size in instances in which a significant deviation exists.

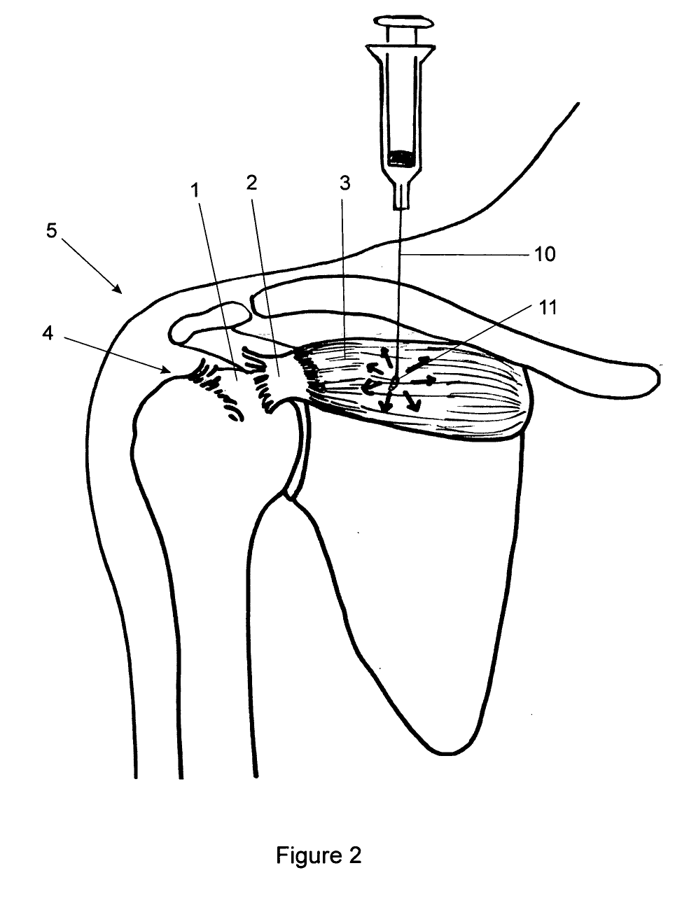

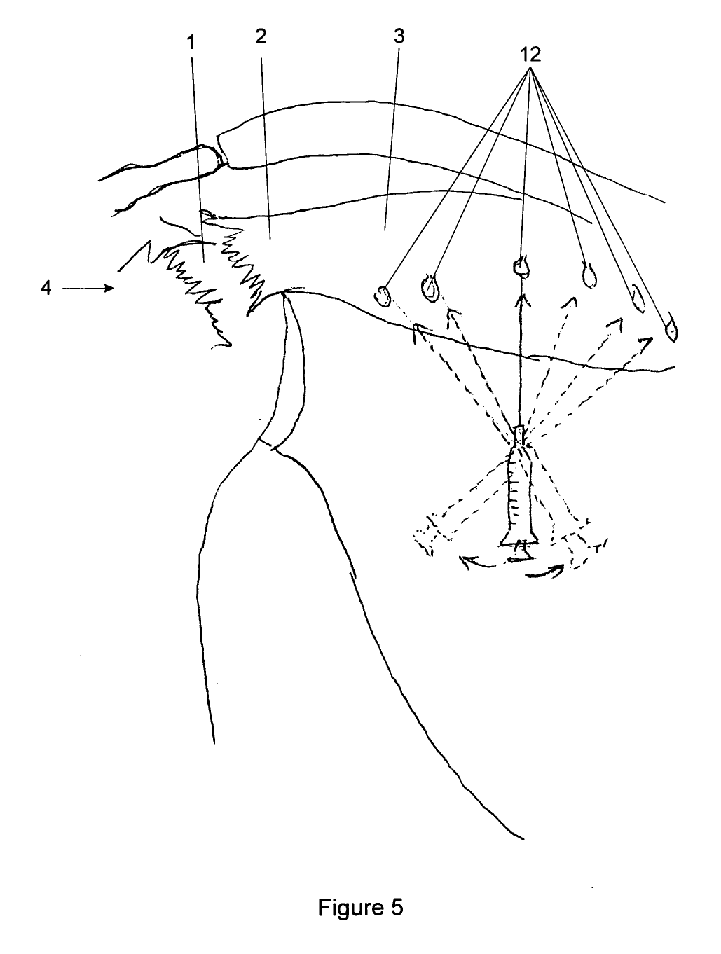

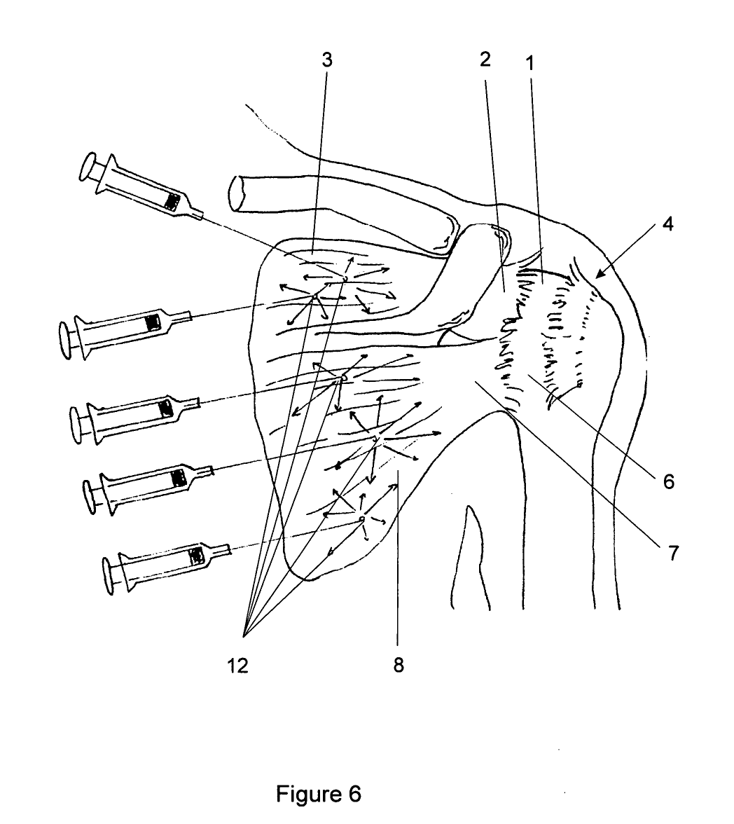

The injection technique is preferably as follows and is indicated by numeral (11) in FIGS. 2 and 3 as a single injection site in respect of each muscle, simply the indicate that the application of the invention. However, whilst conceivably being possible under certain circumstances, the diffusion of the neurotoxin into the muscle would generally be expected to be too slow to be effective from a single injection site. Multiple injection sites (12) as described hereinafter and illustrated in FIGS. 5 and 6 would thus undoubtedly be used in the absence of any special technique of enhancing diffusion into the entire muscle from a single injection site. Trials conducted to date indicate that typically four injection sites would be appropriate to each other muscles although more or less may be employed according to advancing experience as time goes by. The injection procedure can be done with the patient either in a sitting or lateral lying position on the unaffected side.

The supraspinatus and infraspinatus muscles are injected with the botulinum toxin with a needle (10) having a minimum length of 35 mm and a thickness typically between 26 and 30 Gauge. These muscles are easily palpated above and below the spine of the scapula (the bony prominence on the back of the scapula). Cognisance must be taken of the fact that other muscles are more superficial than the supraspinatus and infraspinatus muscles.

The trapezius muscle is superficial to the supraspinatus muscle and the deltoid muscle is superficial to the infraspinatus muscle. Care must therefore be taken to avoid injecting these more superficial muscles with neorotoxin, as it will cause paresis or paralysis of these muscles. Although this will have no long-term deleterious effect, the desired effect on the supraspinatus and infraspinatus muscles will then not be achieved.

Different methods of determining the location of the supraspinatus and infraspinatus muscles can be used:

The first method may generally be considered to be suitable if there is no availability of special equipment such as EMG (electromyography) or ultrasound to localize the target muscle.

A 26 Gauge or thinner needle with a minimum length of 35 mm (about 1⅜ inches) is used. The spine of the scapula is palpated and approximately halfway down the spine a line is drawn perpendicular to the spine in the anterior direction. Measuring 2 cm (about ¾ inch) from the spine, a point on the skin is marked. This is then used as the entry point. The needle is penetrated through the skin, perpendicular to the skin surface, until the bone that forms the bottom of the supraspinatus fossa is reached. This step is necessary to make sure that the needle is not too superficial, and therefore avoids accidental injection of the more superficial Trapezius muscle. The needle is then retracted for approximately 1 cm and the first deposit of neurotoxin is administered (+/−0.2 ml or 0.0012 cubic inches). From this point the technique described above is repeated with administering deposits in a fanwise manner, to ensure that the complete supraspinatus muscle belly has been injected with a total of 3.5 ml (about 0.21 cubic inches) neurotoxin solution.

A similar technique may be used to inject the infraspinatus muscle. The entry point for the needle is approximately halfway down the spine of the scapula and 3 cm (about 1¼ inches down). The needle is inserted perpendicular to the skin and pushed down to the bone. The needle is retracted 1 cm and the first deposit of neurotoxin solution is injected (+/−0.2 ml or 0.0012 cubic inches). In a fanwise manner the complete infraspinatus muscle belly is injected using a total of 3.5 ml (about 0.21 cubic inches) neurotoxin solution (100 units of neurotoxin).

In the instance in which an ultrasonography device is available for use, the target muscles may be identified with a 3.5 Mhz angular transducer. The muscle is then injected using the technique described above. The injection of the neurotoxin solution can be observed on the ultrasound monitor.

Another method to localize the target muscles is the use of electromyography (EMG), but this is generally not practical as most surgeons would not have ready access to such equipment.

The clinical effect of the botulinum toxin is generally noticeable after 48 hours, and has its maximum effect between 1 and 4 weeks after the injection.

The surgery is typically scheduled for approximately 1-2 weeks after the injection treatment. In this application the surgery is carried out by way of arthroscopy or open surgery.

The surgical procedure is usually done under general anaesthesia and involves the following.

During the evaluating or diagnostic arthroscopy that precedes the actual repair, the muscles of the rotator cuff are inspected. Using a nerve stimulator, a conduction needle is placed in the muscle belly of the supraspinatus and infraspinatus and stimulated to record muscle contraction. Such nerve stimulators are standard equipment in operating theatres as they are routinely used by anaesthetists.

The needle is then directed to the suprascapular notch, where the suprascapular nerve is located, and the nerve is stimulated while the muscles are directly viewed with the artroscope. The presence and degree of contraction is recorded.

If paralysis of the supraspinatus and infraspinatus is confirmed, the patient will generally not have to wear an immobilisation sling and is allowed to move the shoulder freely.

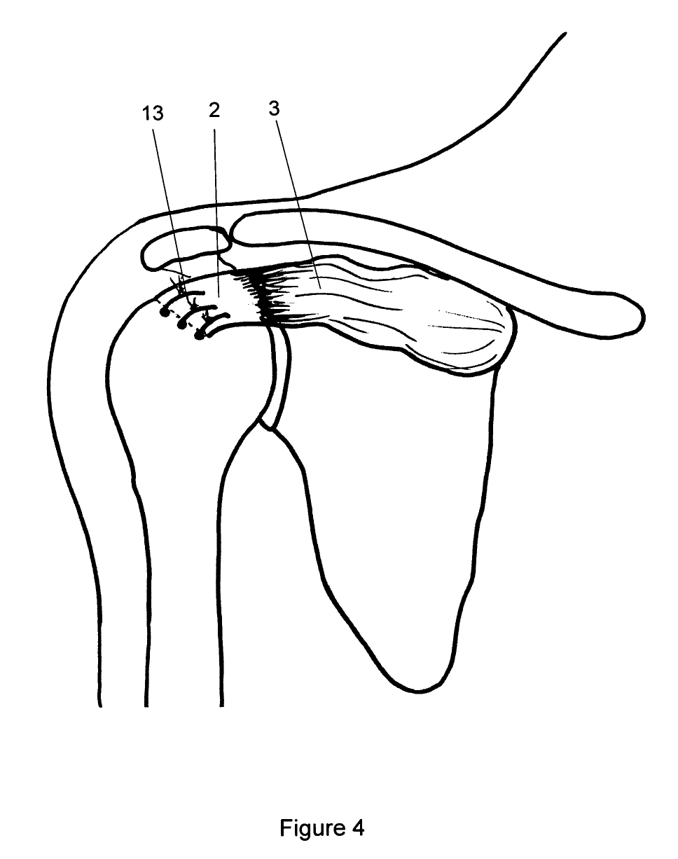

The operation is then conducted in the usual way and the repaired tendon is indicated by numeral (13) FIG. 4.

The patient will then be examined postoperatively according to a standard follow-up schedule that typically includes a 3 week and 6 week postoperative check.

In consequence of the paralysis of the muscles as described above, the pain normally associated with such operations is substantially reduced as is the possibility of the patient causing damage to the repair in consequence of involuntary movements.

It will be understood that the principles of the invention can be applied to other tendon repair surgery and, in particular, it is envisaged that repair of an Achilles tendon could benefit significantly from use of the invention. Other muscles that could benefit from this method are:
the quadriceps muscle of the thigh;
the biceps muscle of the upper arm;
the flexor and extensor muscles of the forearm; and,
the triceps muscle of the upper arm.

In summary, the benefits to patients suffering muscle and tendon ruptures requiring surgical repair by employing the invention are as follows:
1. During the pre-operative period there is excellent pain relief due to the relaxation/paralysis of the injured muscle/tendon.
2. During this pre-operative phase further retraction, shortening and atrophy of the injured structure is minimised.
3. During the surgical procedure the actual repair can be made technically easier for the surgeon due to the fact that the tension in the muscle is diminished and therefore the tendon will reach its insertion site with greater ease.
4. During the post-operative phase the pain is generally greatly diminished due to the reduction in the tension of the repaired structure.
5. The patient is not immobilised as he or she cannot inadvertently contract the repaired muscle/tendon
6. Not requiring immobilisation has an enormous impact in that the patient can, at least a large extent, comfortably continue daily activities, including self-care, hobbies and, most importantly, continue to be economically active.
7. The most important benefit is that the repaired structure is substantially immobilised by temporary selective paralysis, therefore preventing inadvertent contraction and disrupting the repair; a not infrequent event.

1. A method of repairing tendons by surgery comprising:
identifying a tendon to be repaired;
identifying a muscle associated with the tendon and administering to the muscle a dose of a neurotoxin sufficient to effectively cause temporary paralysis of the muscle;
allowing a sufficient period of time after administration of the neurotoxin to elapse to enable said temporary paralysis of the muscle to set in;
performing repair surgery on the relevant tendon; and
optionally, if necessary, treating said muscle with one or more additional doses of neurotoxin to maintain temporary paralysis for at least an initial period of rehabilitation.

2. The method of claim 1, wherein the neurotoxin is injected into the muscle.

3. The method of claim 2, wherein the neurotoxin is injected into the muscle at multiple injection sites.

4. The method of claim 1 in which the neurotoxin is a slow acting neurotoxin.

5. The method of claim 4, wherein the neurotoxin is a suitable botulinum toxin, and wherein said sufficient period of time is of the order of 7 to 21 days and no additional doses of neurotoxin are administered.

6. The method of claim 5, wherein said sufficient period of time is about 10 to 14 days.

7. The method of claim 1, wherein the degree of paralysis of the muscle is tested immediately prior to performing the repair surgery and, in the event of a good paralysis being detected, then allowing the patient to undergo a period of rehabilitation without any immobilization of the relevant limb.

8. The method of claim 1, wherein the tendon is a rotator cuff tendon selected from those associated with the supraspinatus and infraspinatus muscles.

9. The method of claim 8, wherein both of the supraspinatus and infraspinatus muscles are subjected to temporary paralysis preparatory to conducting the surgery irrespective of the fact that only one of them is to be repaired.

10-11. (canceled)
